(19) United States
(12) Patent Application Publication
(10) Pub. No.: US 2016/0120441 A1
(43) Pub. Date: May 5, 2016

(54) Portable Device for Direct Nasal Respiration Measurement

(71) Applicant: Xiaoran Zhu, Crozet, VA (US)

(72) Inventor: Xiaoran Zhu, Crozet, VA (US)

(21) Appl. No.: 14/530,827

(22) Filed: Nov. 2, 2014

(51) Int. Cl. A61B 5/097; A61B 5/00; A61B 5/087

(52) U.S. Cl. CPC A61B 5/097; A61B 5/087; A61B 5/0004; A61B 5/486; A61B 5/6819; A61B 5/742; A61B 2560/0425; A61B 2562/164; A61B 2562/245; A61B 2560/0214; A61B 2562/028; A61B 2560/0443

(57) ABSTRACT

A portable and wireless connected device, comprising a breathing channel part, a sensor box, a nose piece and a cover, allows measuring air flow of respiration through each nostril. All components are in conjunction with other parts through quick assembly mechanisms. Multiple parameters including air flow speed, volume and respiration rate are determined by analyzing sensors data in real time, thereby allowing diagnosis, monitoring and interactive breathing training in patients with pulmonary diseases.

### CROSS REFERENCE TO RELATED APPLICATIONS

The present application claims priority under 35 §119(e) from U.S. Provisional Application Ser. No. 61/962,135, filed Nov. 1, 2013, entitled “A Portable Nasal Spirometer for Testing Certain Pulmonary Diseases;” the disclosure of which is hereby incorporated by reference herein in its entirety.

### FIELD OF THE INVENTION

The present invention relates to human respiration measurement. More specifically, the invention relates to a measurement of respiration through each individual nostril simultaneously with a portable and wireless device.

### BACKGROUND OF THE INVENTION

Medical treatment, such as an inhaler, can alleviate the symptoms of pulmonary diseases; however excessive use of medicine may cause serious side effects. Other trials such as breathing exercise have been proved to significant reduce the need for inhaler usage among many patients. For these exercises, the key to success is to strictly follow an individually tailored training plan. A real time measurement of nasal respiration is of particular benefit to pulmonary disease patients both for diagnosis and therapy training.

A majority of respiration measurement devices are used in the hospital setting. Patients must go to a hospital to have their spirometry data collected. For this routine practice, spirometry data is abstained exclusively through mouth breathing.

In some research experiments, nasal spirometry has been studied by using a portable mouth spirometer through a nose adapter. Respiration of two nostrils were then measured separated with the modified device and the results were used for septal deviation diagnosis.

In other research studies, various MEMS flow sensors were proposed for respiratory measurement.

For one type of method, sensor bands attached to an object's chest and abdomen were used to measure respiration. Since this uses indirect measurement, sensor calibration is highly recommended before use to obtain more accurate results. Furthermore, object posture and body movement may cause substantial errors in the measurement.

For one type of method, it attaches a mask to an object wherein induced current is generated during respiration. The value of induced current is proportional to the air flow rate. However, the proposed current generation device has a size of a nostril which makes it extremely difficult for manufacturing. Furthermore, a device with a moving fan will be difficult to work in a moist environment.

### SUMMARY OF THE INVENTION

An aspect of an embodiment of the present invention provides a portable and wireless device suitable for accurate respiration measurement through nose instead of mouth.

An aspect of an embodiment of the present invention provides a nasal respiration measurement device, comprising a breathing channel part, a sensor box, a nose piece and a cover. All components are in conjunction with other parts through quick assembly mechanisms. Upon assembly, the breathing channel part and the nose piece create smooth, seamless and airtight breathing channels for guiding the respiration air flow during operation. Upon assembly, the breathing channel part and the sensor box form smooth, seamless and airtight bypasses wherein the sensors are located.

An aspect of an embodiment of the present invention provides a breathing channel part for the device. The breathing channel part may comprise breathing channels, protruded connectors, and orifice structures. The breathing channel part may be washable for disinfection and sanitizing.

An aspect of an embodiment of the present invention provides a nosepiece for the device. The nosepiece may be made of silicone, rubber or other substantially soft materials. The nosepiece may be disposable or washable for disinfection and sanitizing.

An aspect of an embodiment of the present invention provides a sensor box for the device. The sensor box may comprise protrusions from the top surface with cavities for sensors mounting, MEMS air flow sensors, sensor amplifier circuits, and a wireless data acquisition module. The MEMS air flow sensors have no electrical contact pad on surface and can be sanitized by wiping with an alcohol wipe.

These and other advantages and features of the invention disclosed herein, will be made more apparent from the description, drawings and claims that follow.

### DESCRIPTION OF THE DRAWINGS

The accompanying drawings, which are incorporated into and form a part of the specification, illustrate one or more embodiments of the present invention and, together with the description herein, serve to explain the principles of the invention. The drawings are only for the purpose of illustrating select embodiments of the invention and are not to be construed as limiting the invention. In the drawings:

FIG. 1 is a schematic diagram illustrating the device.

FIG. 2 provides a perspective view and a cross-sectional view showing an assembled prototype of the device according to an embodiment of the present invention.

FIG. 3A and FIG. 3B provide multiple views of the sensor box that contains air flow sensors and circuits.

FIG. 4A illustrates the side view and bottom view of the breathing channel part of the device assembly. FIG. 4B is the prospective cross-sectional view of the part.

FIG. 5 is the cross-sectional view and side view of the nosepiece of the device assembly.

FIG. 6 illustrates the cover of the sensor box.

FIG. 7 provides schematic illustrations of an exemplary embodiment of an air flow sensor.

FIG. 8 provides a schematic diagram of the low battery indicator circuit to be used in conjunction with the present invention.

FIG. 9 provides a schematic diagram of the amplifier circuit for flow and temperature sensing to be used in conjunction with the present invention device.

FIG. 10A illustrates a graphical image of a simulation for pressure distribution in the breathing channel during respiration. FIG. 10B is a plot of pressure change along line M-N. FIG. 10C illustrates a graphical image of a simulation for flow speed in the breathing channel during respiration. FIG. 10D is a plot of speed change along line M-N.

FIG. 11 provides a graphical illustration of measuring the control pause of respiration according to one embodiment of the present invention.

FIG. 12A and FIG. 12B provide a graphical illustrations of measuring the breathing patterns of respiration according to one embodiment of the present invention.

FIG. 13 provides a graphical illustration of measuring the forced vital capacity of respiration according to one embodiment of the present invention.

FIG. 14A and FIG. 14B provide a graphical illustrations of detecting nasal septal deviation according to one embodiment of the present invention.

FIG. 15 provides a graphical illustration of nasal spirometry measurement according to one embodiment of the present invention.

### DETAILED DESCRIPTION OF THE INVENTION

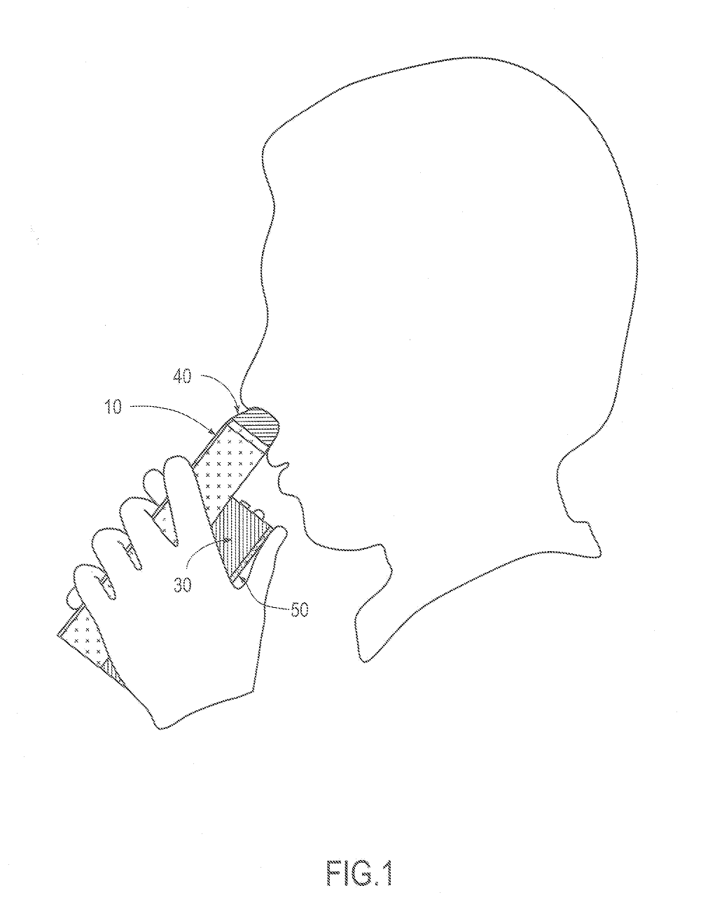

The present invention is of a portable device that allows air flow of respiration through each nostril to be directly measured. Multiple parameters including air flow speed, volume and respiration rate can be determined by analyzing the data in real time, thereby allowing for the diagnosis of pulmonary diseases such as asthma and COPD. On the other hand, this real time information would be very beneficial for pulmonary disease patients performing interactive breathing exercise. FIG. 1 is a schematic diagram illustrating the device and method of the invention as one embodiment of the present invention. The device comprises a breathing channel part 10, a sensor box 30, a nosepiece 40 and a cover 50. As one embodiment of the invention, all parts are assembled by means of direct insert or clip-on.

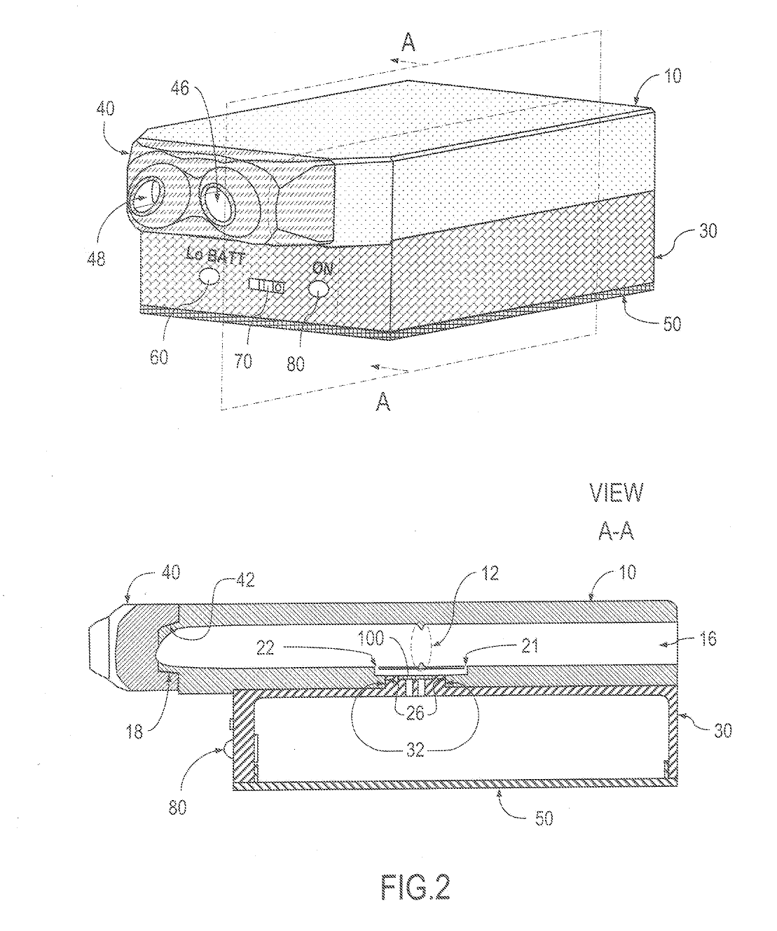

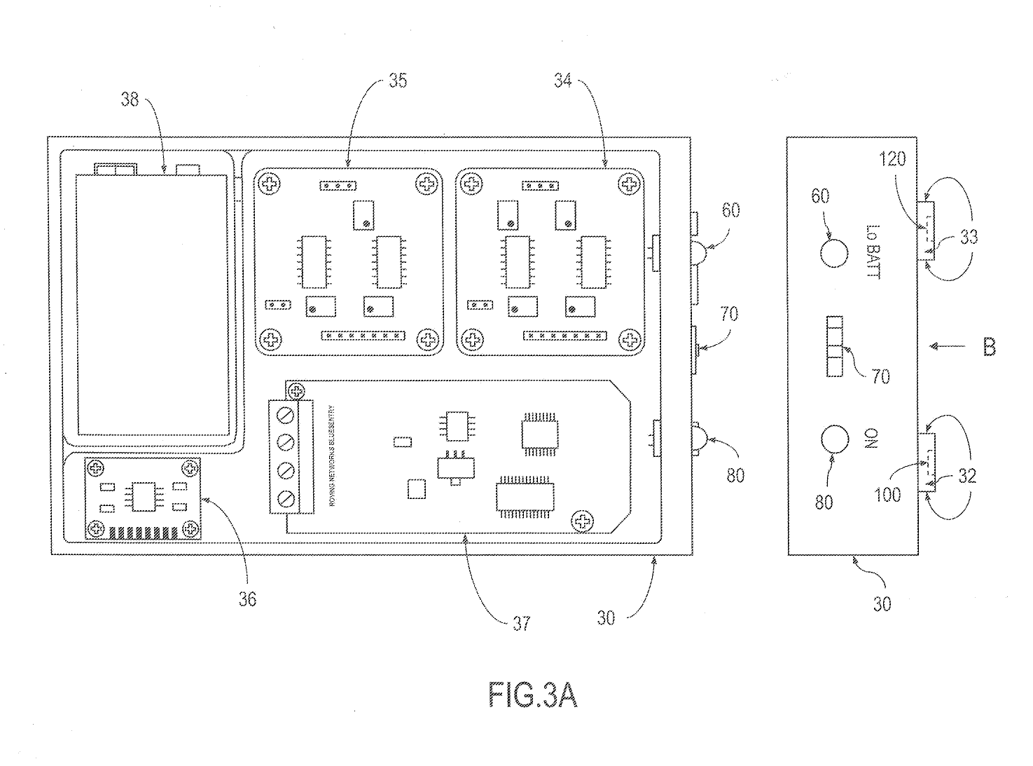

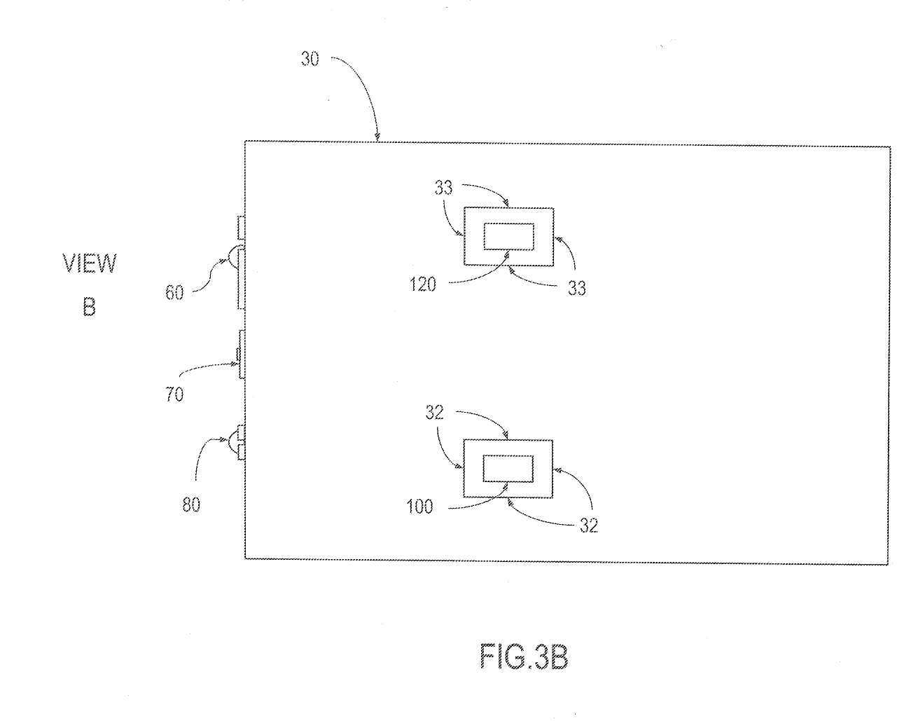

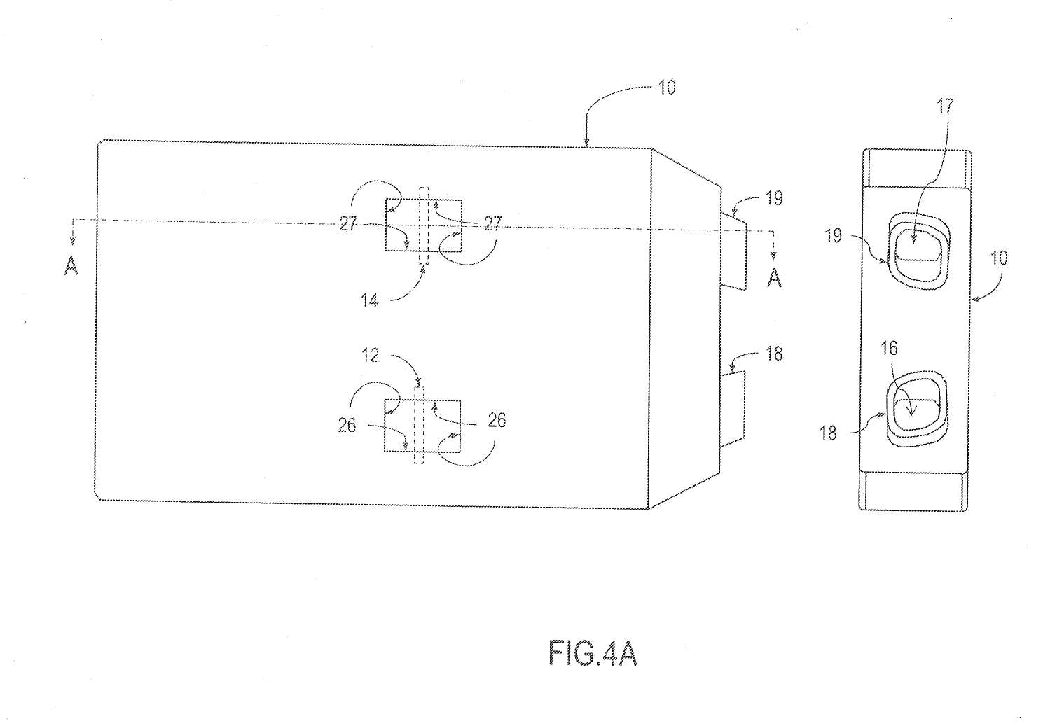

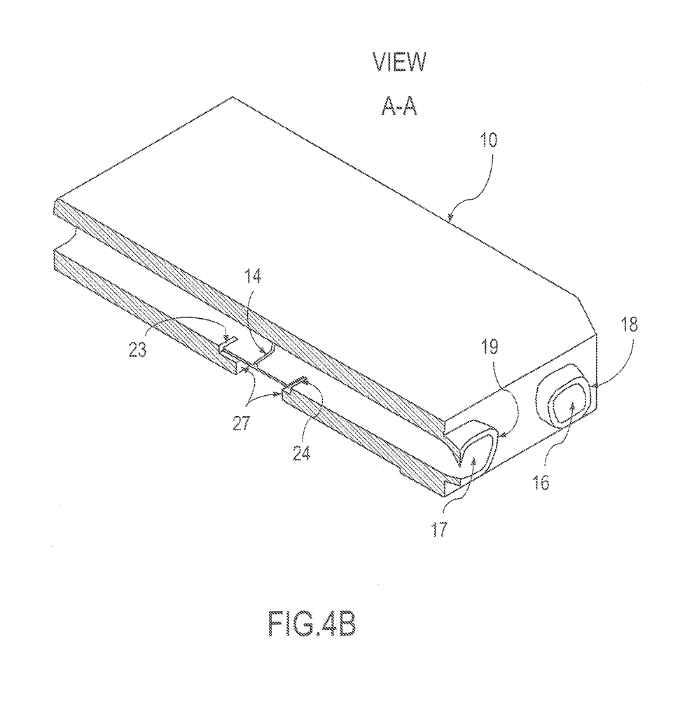

FIG. 2 provides a perspective view and a cross-sectional view showing an assembled prototype of the device according to one embodiment of the present invention. The breathing channel part 10 is attached to the sensor box 30 through a means of insert in connection 26/32 (and 27/33 shown in FIG. 3B and FIG. 4A). The nosepiece 40 is attached to the breathing channel part 10 by a clip-on connection 18/42 (19/43 shown in FIG. 4B and FIG. 5), forming a continuous breathing airways 16-46 for the right nostril (17-48 for the left nostril). Airflow of respiration is guided through the airway 16-46 during the measurement. A small portion of the air flow goes through slit 21 and slit 22 built in the breathing channel part 10. The quantity of bypassed portion of air flow is determined by the orifice 12 built in the breathing channel part 10. The speed of the air flow is measured by the air flow sensor 100 mounted on the sensor box 30. A green LED diode 80 is mounted in the sensor box 30, indicating the device is on. A red LED diode 60 is mounted in the sensor box 30, indicating low battery voltage. A switch 70 is mounted in the sensor box 30 for turning the device on and off. Finally, the cover piece 50 is clipped on the sensor box 30 for further protection.

Referring to FIG. 3A, which provides detailed drawings of the sensor box 30 that contains air flow sensors and circuits. Two air flow sensors 100, 120 are mounted on two rectangular protrusions defined by surface 32 and 33. Two air flow sensors 100/120 are installed in each of the protrusions to keep the surfaces flat. Sensor signal amplifier circuits 34 and 35 are packaged in the sensor box 30 and are wire-connected to left air flow sensor 120 and right air flow sensor 100, respectively. A wireless data acquisition module 37 is packaged in the sensor box 30 and is wire-connected to the outputs from the sensor signal amplifier circuits 34 and 35. One example of choice for the wireless data acquisition module 37 is the BlueSentry from Roving Networks Inc., which communicates with a computer or other devices through Bluetooth technology. A 9 volt battery 38 is packaged in the sensor box 30 as the power supply. A power indicator circuit 36 is installed in the senor box 30 monitoring battery status. When the voltage of the battery 38 is lower than 6.0 v, the red low battery indicator LED diode 60 will turn on, prompting the need for battery replacement. FIG. 3B is another view of the sensor box 30 showing the locations of the air flow sensors.

Referring now to FIG. 2, after assembly, sensor 100 (120) will be located in the middle of the breathing airways 16-46 (17-48) crosswisely as well as lengthwisely.

Referring now to FIG. 4, which depicts detailed structure of the breathing channel part 10. In FIG. 4A, two openings defined by surface 26 and 27 are constructed in the bottom surface of the breathing channel part 10. The openings are paired with the protrusions of the sensor box 30 forming an insert in connection between 10 and 30. Structure 18 and 19 are designed in order to form interference fittings of 18-42 and 19-43 with the nosepiece 40. Channel 16 and 17 are parts of the breathing airways which transition smoothly across the breathing channel part 10. FIG. 4B is the perspective cross-sectional view of part 10, illustrating detailed structures in the channel 17. In the middle of the channel 17 is the orifice 14 for air flow flux distribution. During testing, large portion of the air flow goes through the main channel, while a small portion of the air flow bypasses through slit 23 and 24 where the air flow sensor 120 located.

After assembly between the breathing channel part 10 and the sensor box 30, referring now to the cross-section view in FIG. 2, a bypass channel from slit 21 to slit 22 is formed.

Referring now to FIG. 5, which provides detailed drawings of the nosepiece 40. According to one embodiment of the present invention, the nosepiece 40 is made of soft silicone material. The protrusions 45 and 47 are in conjunction with right nostril and left nostril during operation. The chamber 42 and 43 are created to form interference fittings with 18 and 17 in the channel part 10. Channel 46 and 48 are conjunction with 16 and 17 forming seamless breathing channels of the whole assembly.

Referring now to FIG. 1, during respiration measurement, the nose piece 40 is pushed against the nose with a moderate pressure in order to seal the air flow.

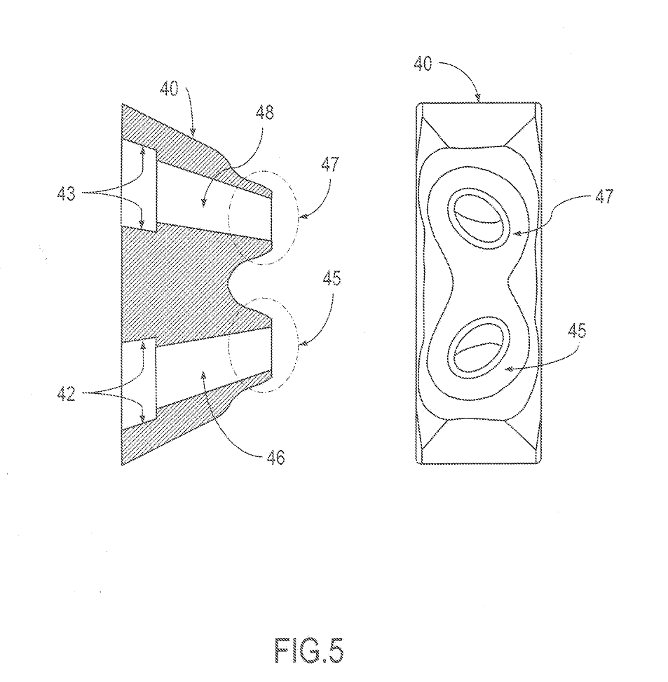

Referring now to FIG. 6, which provides views of the cover 50. The cover 50 provides protection to the electronics packaged in the sensor box 30.

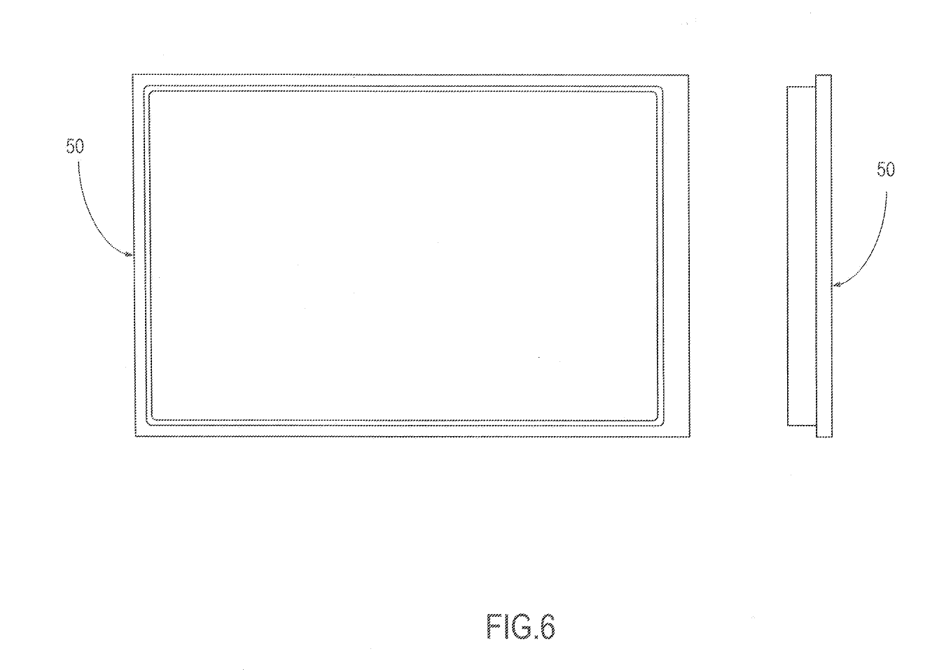

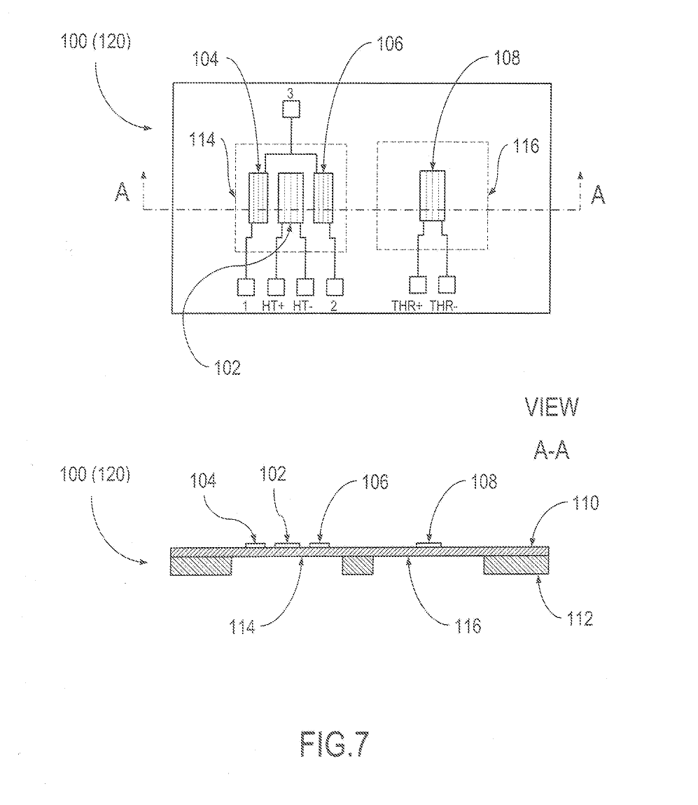

Referring now to FIG. 7, which provides a schematic diagram of a MEMS air flow sensor 100 (120). Freestanding diaphragm 114 and 116 are microfabricated on substrate 112. One heating element 102 and three thermal sensing elements 104, 106 and 108 are microfabricated in a configuration depicted in FIG. 7.

Referring now to FIG. 3A and FIG. 3B, the air flow sensors 100, 120 are embedded in the sensor box 30 and wire-connected to the sensor signal amplifier circuit 34 or 35 to measure the speed of air flow.

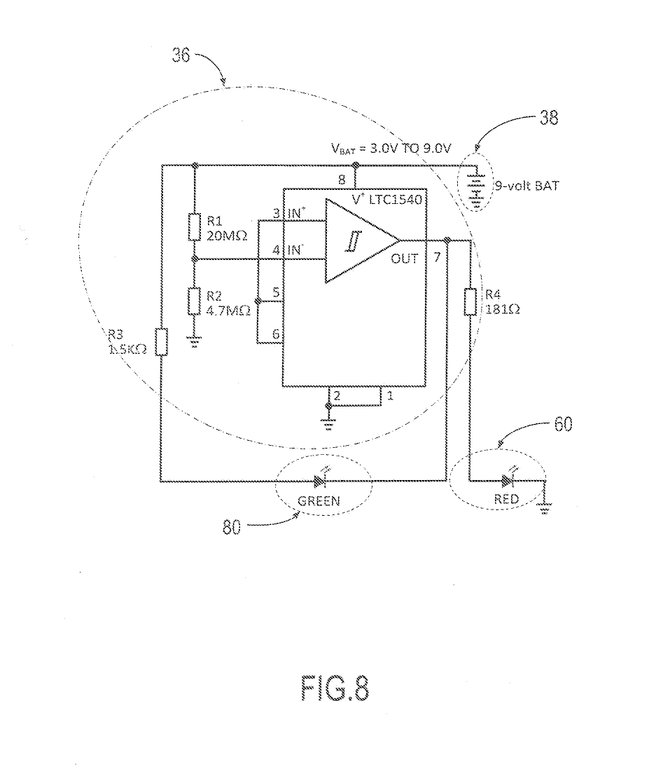

FIG. 8 provides a schematic illustration of the low battery indicator circuit 36. This circuit is powered by the battery 38 while providing battery capacity information. The output signals are connected to the green diode LED 80 and the red LED diode 60.

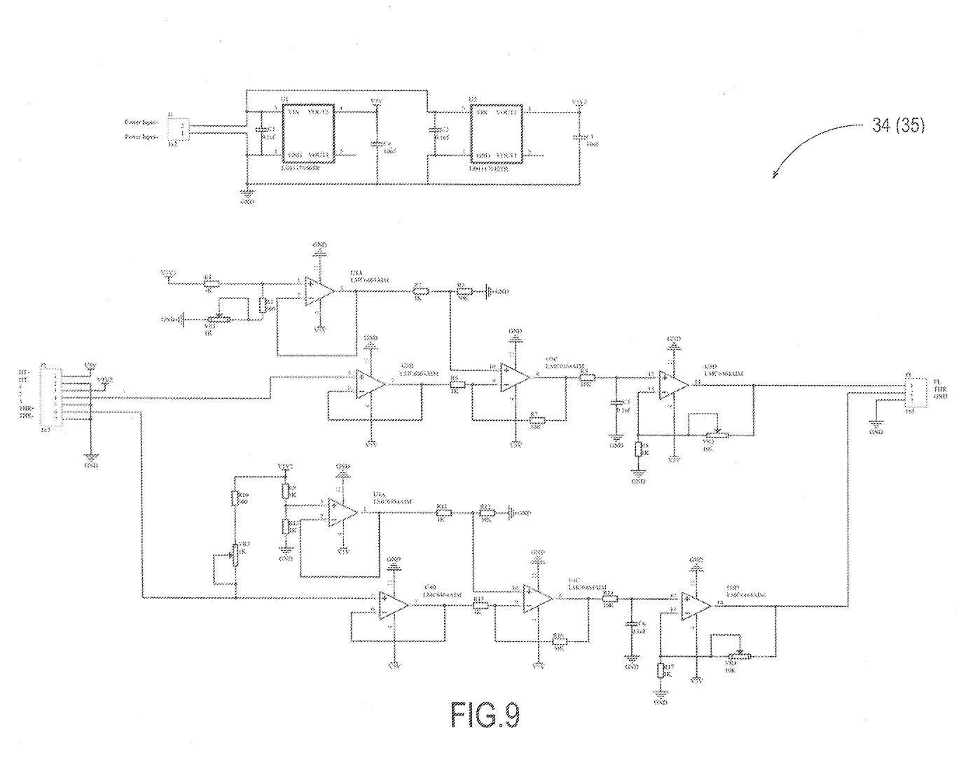

Referring now to FIG. 9, which provides a schematic diagram of the sensor signal amplifier circuit 34 (35).

Although the invention has been described in detail with particular reference to these preferred embodiments, other embodiments can achieve the same results. Variations and modifications of the present invention will be obvious to those skilled in the art from a consideration of this disclosure or practice of the invention disclosed herein. Consequently, it is not intended that this invention be restricted solely to the specific embodiments disclosed herein, but that it covers all modifications and alternatives coming within the true scope and spirit of the invention.

### EXAMPLES AND EXPERIMENTAL RESULTS

The examples and experimental results provided below are not intended to be limiting to the scope of the invention and serve for illustration only.

### Example No. 1

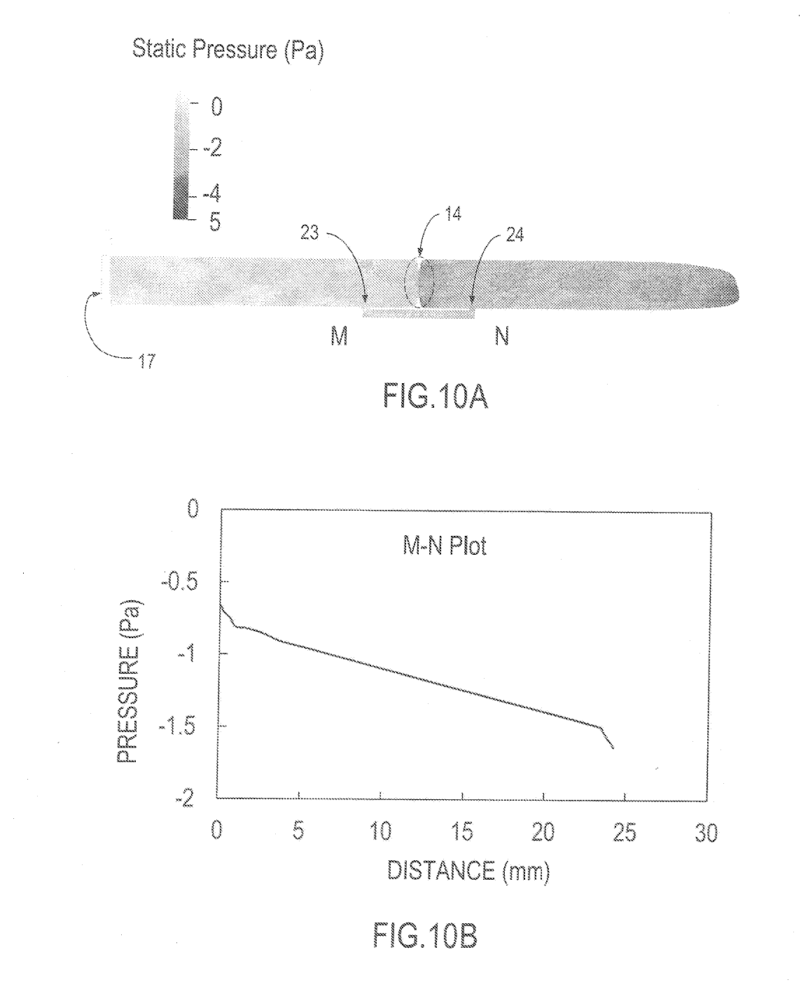

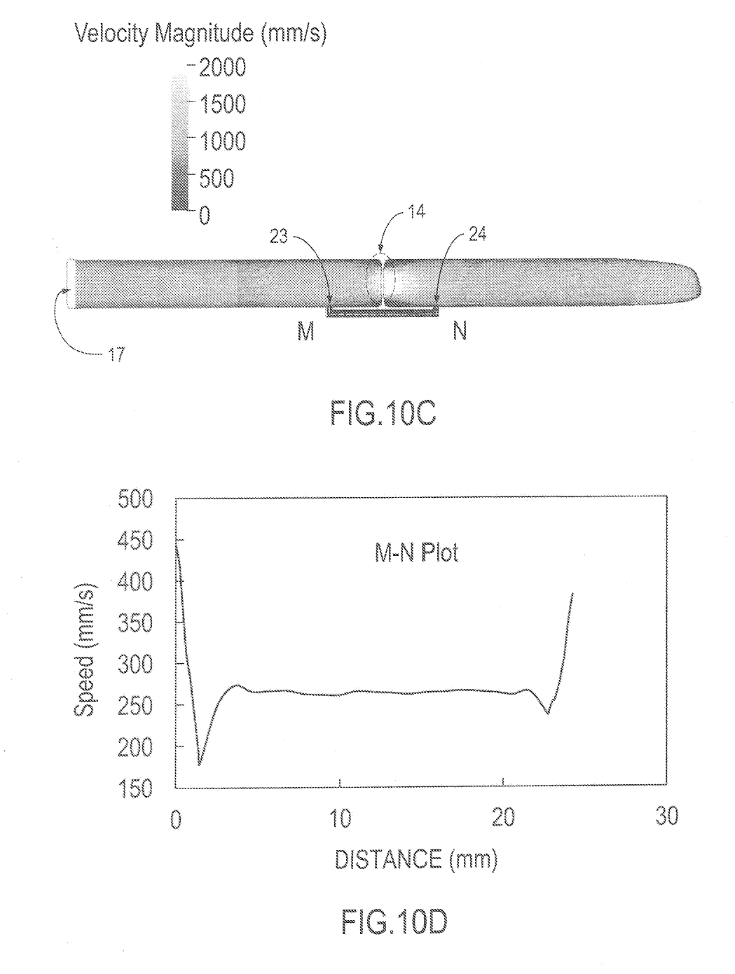

An experiment was carried out to simulate the pressure distribution in the breathing channel 17 during inhalation through the present invention device. According to one embodiment of the sensing device, pressure difference caused by the orifice 14 enables small portion of the air flow to pass through the slits 23, 24 and therefore reach the air flow sensor. In this simulation, we set the initial condition for the breathing flow rate to be steady state at 0.1 L/s. The result shown in FIG. 10A illustrates the existence of pressure gradient in the bypass channel. In this case, the pressure difference between slit 23 and slit 24 is about 1 Pa. FIG. 10B shows a steady pressure gradient established in the bypass channel along line MN. The pressure gradient is about 30 Pa/m.

FIG. 10C shows a computational fluid dynamics simulation of velocity magnitude of the air flow in the breathing channel 17 for inhalation through the present invention device. Air travels much slower in the bypass channel than in the main channel as suggested by an embodiment of the present invention. FIG. 10D is a plot of air flow speed along line MN. In the middle section of the bypass channel air flow speed is constant, at about 270 mm/s.

It should be appreciated that the orifice is intended as one embodiment for air flow bypassing in the breathing channels. Other variations or modifications can be practiced for the same purpose without departing from the spirits of the present invention.

### Example No. 2

Another experiment was carried out to measure the Control Pause commonly used by Buteyko breathing practitioners as a body oxygen level indicator. FIG. 11 illustrates the process of the measurement. The longest breath holding time 140 between normal breathing patterns in FIG. 11 is called Control Pause. According to an embodiment of the present invention, flow information is wirelessly sent to a display device such as computer, tablet, smart phone or other electronic device to show the graph. By using the present invention device, any incorrect measurement could be visually captured in the graph and should be avoided in practice.

### Example No. 3

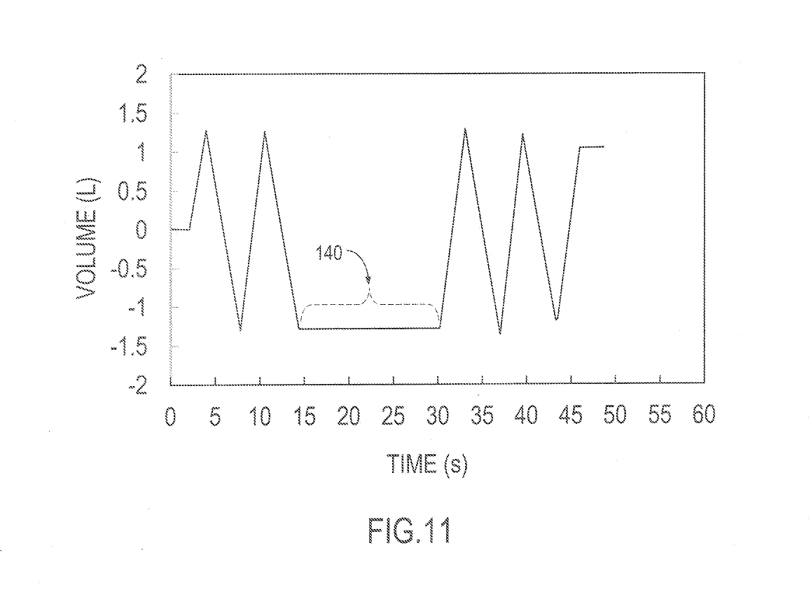

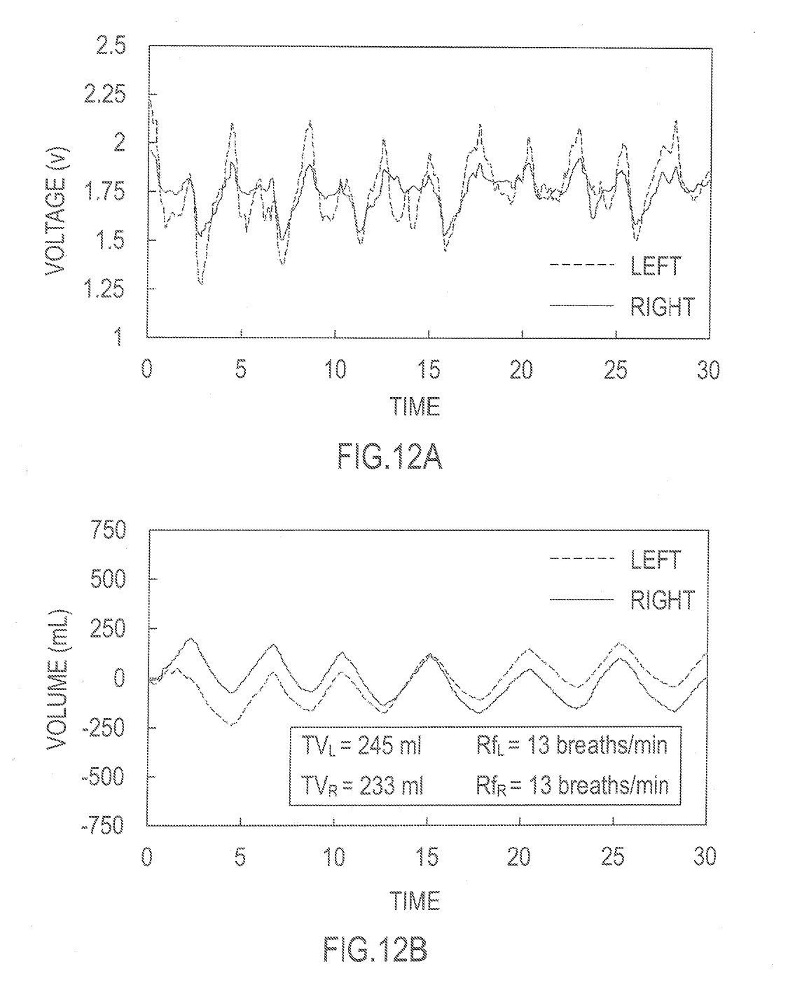

Another experiment was carried out to measure the air flow through each nostril during normal breathing. Original signals of each air flow sensor were collected and sent to a computer, tablet, smart phone or other electronic device through a wireless module. FIG. 12A shows an example of the original air flow sensor signals. According to one embodiment of the present invention, the signal was collected by a Bluetooth data acquisition module and sent to a computer. A computer program, based on LabVIEW software, received data and subsequently processed them. The data was dynamically displayed on screen. The signal for the left and the right nostrils are in the same phase and have similar strength. Further the program can convert the original data into volume. FIG. 12B shows the result from data processing, from which the program concluded that the inhale volume and exhale volume for each nostril as well a breathing frequency. The average inhale and exhale volume (tidal volume) for left nostril and right nostril are 245 mL and 233 mL, respectively. The total tidal volume is 478 ml. The respiratory frequency or respiratory rate is about 13 breaths per minute.

### Example No. 4

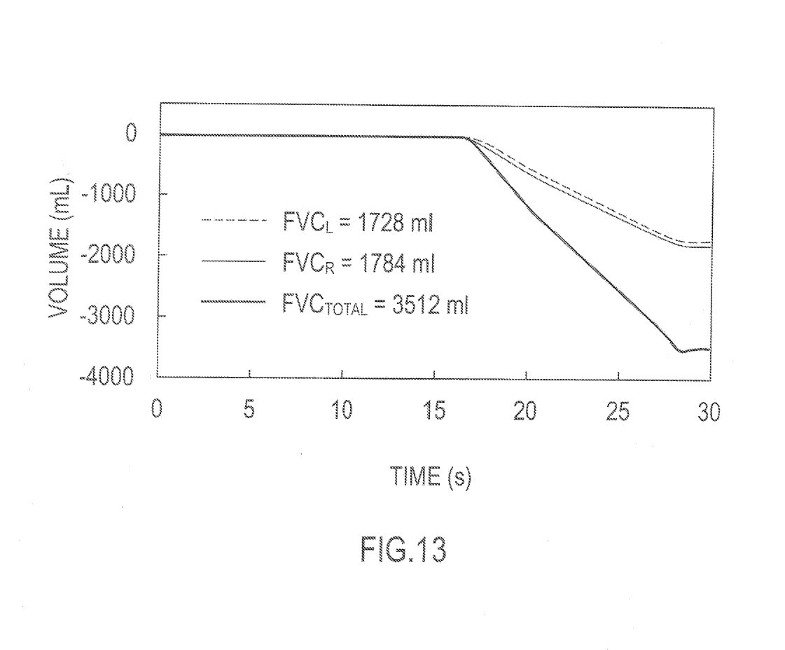

Another experiment was carried out to measure the forced vital capacity (FVC) of the lung. With the present invention device, forced vital capacity through nostrils can be measured precisely. FIG. 13 illustrates the measurement results. Forced vital capacity through left nostril is 1728 mL. Forced vital capacity through right nostril is 1784 mL. Overall forced vital capacity is 3512 mL. The length of exhale breath during the test is about 12 s.

### Example No. 5

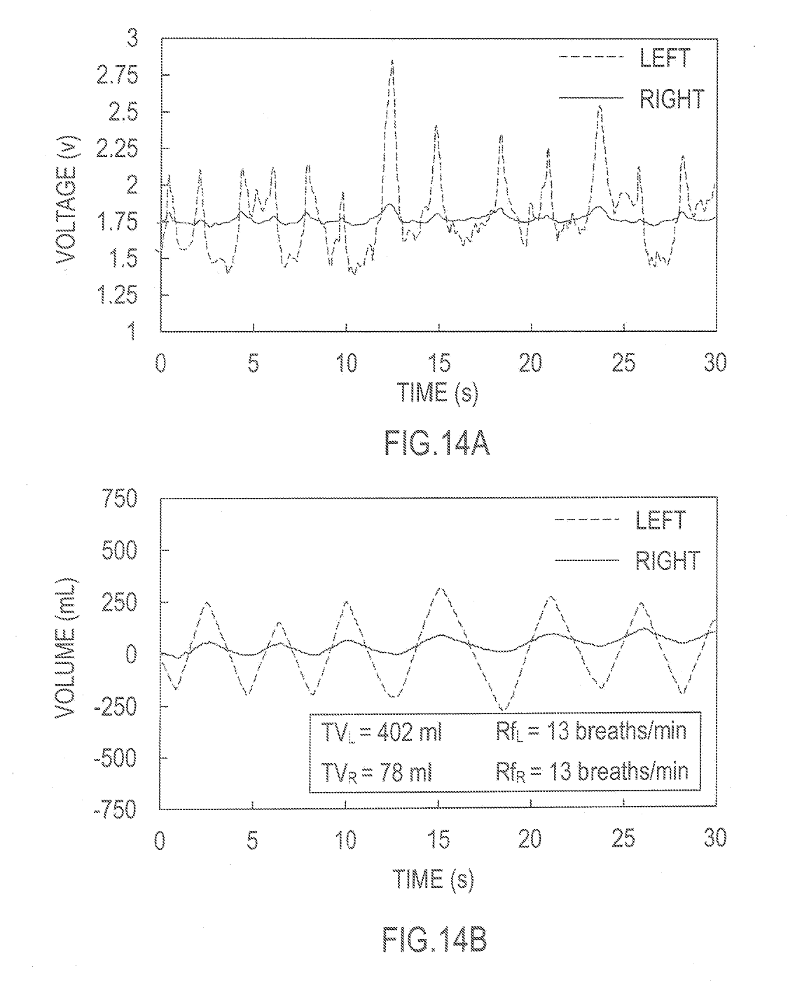

Another experiment was carried out to compare volume of air flow through each nostril during normal breathing, quantitatively. According to one embodiment of the present invention, the device can be used for diagnose of nasal septal deviation. In this case, original signals of each air flow sensor were collected and sent to a computer, tablet, smart phone or other electronic device with a wireless module. According to another embodiment of the present invention, the signal was collected by a Bluetooth data acquisition module and sent to a computer. A computer program, based on LabVIEW software, received data and subsequently processed it. The data was dynamically displayed on screen. FIG. 14A indicates that the left sensor has a significant higher signal than the right sensor. From FIG. 14B, information shows that tidal volume from left nostril is 402 mL and those from right nostril is 78 mL, indicating a strong nasal septal deviation.

### Example No. 6

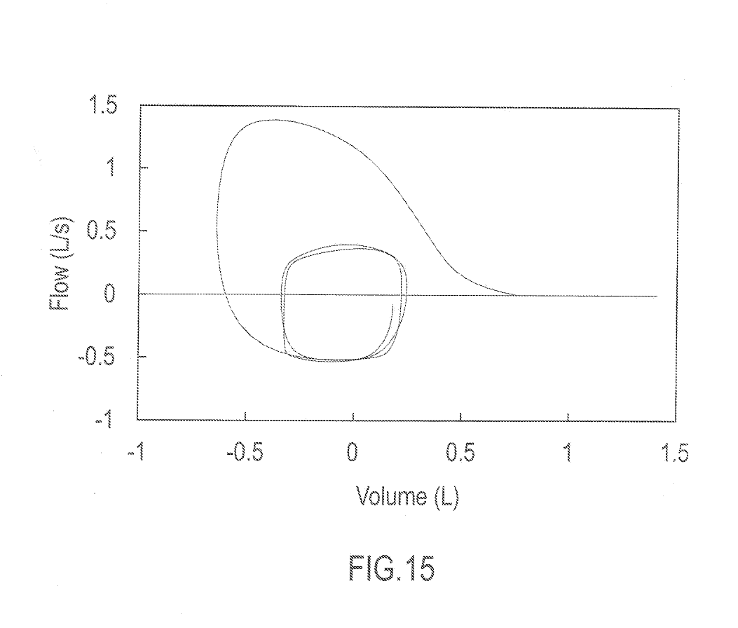

Another experiment was carried out to conduct a nasal spirometry test. FIG. 15 shows a flow speed and volume graph of a single nostril during nasal spirometry test. The information extracted from the curve in this graph is especially beneficial for clinical diagnosis of asthma and other pulmonary diseases.

### CITATIONS

1. U.S. Pat. No. 8,636,671 B2, Yong Won Jang, “Wearable Respiration Measurement Apparatus”, Jan. 28, 2014.

2. U.S. Pat. No. 6,579,242 B2, Tuan Bui, et al., “Method and Apparatus for Providing Patient Care”, Jun. 17, 2003.

3. U.S. Pat. No. 8,731,646 B2, Halperin, et al., “Prediction and Monitoring of Clinical Episodes”, May 20, 2014.

4. U.S. Pat. No. 8,728,001 B2, Larence A. Lynn, “Nasal Capnographic Pressure Monitoring System”, May 20, 2014.

5. U.S. Pat. No. 8,128,575 B2, Raffaele Dellaca, et al., “System and Method for the Automatic Detection of the Expiratory Flow Limitation”, Mar. 6, 2012.

6. U.S. Pat. No. 7,828,742 B2, In Kwang Lee, et al., “Method and System of Monitoring Respiratory Signal by Radio”, Nov. 9, 2010.

7. U.S. Pat. No. 7,635,339 B2, Karl Harnoncourt and Christian Buess, “Method for Non-cooperative Lung Function Diagnosis Using Ultrasound”, Dec. 22, 2009.

8. U.S. Pat. No. 7,094,206 B2, Andrew Hoffman, “System for Measuring Respiratory Function”, Aug. 22, 2006.

9. U.S. Pat. No. 6,723,055 B2, Andrew Hoffman, “System for Measuring Respiratory Function”, Aug. 20, 2004.

10. U.S. Pat. No. 5,333,106, Ted W. Lanpher, et al., “Apparatus and Visual Display Method for Training in the Power Use of Aerosol Pharmaceutical Inhalers”, Jul. 26, 1994.

11. U.S. Pat. No. 4,777,962, “Method and Apparatus for Distinguishing Central Obstructive and Mixed Apneas by External Monitoring Devices Which Measure Rib Cage and Abdominal Compartmental Excursions During Respiration”, Herman Watson, et al., Oct. 18, 1988.

12. Frank Hedrich, et al., “Thermal Flow Sensors for MEMS Spirometric Devices”, Sensors and Actuators A 162 (2010) 373-378.

13. Chia-Ling Wei, Chien-Fu Lin, and I-Ta Tseng, “A Novel MEMS Respiratory Flow Sensor”, IEEE Sensors Journal (10) 2010 16-18.

14. P. J. Cuddihy and R. Eccles, “The use of nasal spirometry as an objective measure of nasal septal deviation and the effectiveness of septal surgery”, Clinical Otolaryngology & Allied Sciences (28) 2003 325-330.

15. Junaid Hanif, et al., “Use of a Portable Spirometer for Studies on the Nasal Cycle”, American Journal of Rhinology (5) 2001 303-306.

16. D. Graham Roblin, et al., “Normal Range for Nasal Partitioning of Airflow Determined by Nasal Spirometry in 100 Healthy Subjects”, American Journal of Rhinology (17) 2003 179-183.

We claim:

1. A portable device for direct nasal respiration measurement, the said device comprising:
a breathing channel part, wherein respiration air flow is guided through and portioned;
a sensor box to house air flow sensors, a power source, electronic circuits and a wireless communication unit;
a nosepiece with one end in communication with nostrils, the other end in clip-in conjunction with said breathing channel part; and
a cover for protection.

2. The device of claim 1, wherein the said device measures respiration information of each individual nostril separately.

3. The device of claim 1, wherein the said device wirelessly sends respiration information to a computer, tablet, smart phone or other electronic device for data analysis and graph display.

4. The device of claim 3, where said graph is used for guiding breathing rehabilitation exercise.

5. The device of claim 1, wherein said breathing channel part is made of metal, plastics or other essentially rigid materials.

6. The device of claim 1, wherein said sensor box is made of metal, plastics or other essentially rigid materials.

7. The device of claim 1, wherein said nosepiece is made of silicone, rubber or other substantially soft materials.

8. The device of claim 1, wherein said cover is made of metal, plastics or other essentially rigid materials.

9. The device of claim 1, wherein said breathing channel part comprises:
two breathing channels;
two protruded connectors at the end of each breathing channel in conjunction with said nosepiece;
an orifice structure in each of the channels; and
an air flow bypass in each of the channels.

10. The breathing channel part of claim 9, wherein said air flow bypass comprises two slits from the inner wall, a narrow channel and an opening through bottom surface.

11. The device of claim 1, wherein said breathing channel part is washable for disinfection and sanitizing.

12. The device of claim 1, wherein said sensor box comprises:
two protrusions from the top surface with cavities for sensors mounting;
two MEMS air flow sensors to convert the breathing air flow into electrical signal;
two air flow sensor amplifier circuit s to amplify said MEMS air flow sensors signal;
a wireless data acquisition module to collect and send said MEMS air flow sensors data to a computer or a portable electronic device;
a low battery indicator circuit;
a device power on LED;
a low battery status LED;
a battery; and
a switch to turn on and off the said device.

13. The sensor box in claim 12, wherein said MEMS air flow sensors are mounted in cavities of said protrusions and form flat surfaces.

14. The sensor box in claim 12, wherein shape of said protrusion is a cuboid or a cylinder or any prism.

15. The sensor box in claim 12, wherein said MEMS air flow sensors have no electrical contact pad on surface and can be sanitized by wiping with an alcohol wipe.

16. The sensor box in claim 12, wherein said wireless data acquisition module is a Bluetooth module with a function of data acquisition or a Wi-Fi module with a function of data acquisition.

17. The device of claim 1, wherein said nosepiece comprises two protrusions in conjunction with nostrils during operation, two channels and two chambers.

18. The device of claim 1, wherein said nosepiece is disposable or washable for disinfection and sanitizing.

19. The device of claim 1, wherein said nosepiece is connected with said breathing channel part by a clip-in or other quick assembly method, forming two seamless, smooth and airtight connections.

20. The device of claim 1, wherein said breathing channel part is connected with said sensor box with an insert-in or other quick assembly connection, forming seamless, smooth and airtight bypass channels.
